(19) United States
(12) Patent Application Publication
(10) Pub. No.: US 2006/0035962 A1
(43) Pub. Date: Feb. 16, 2006

(54) Method and compositions for preparing a compound using a benzoylating agent essentially free of ring chlorination

(75) Inventors: James H. Johnson, Merrimac, MA (US); Barry J. Hand, Acton, MA (US); Rex T. Gallagher, Beverly, MA (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Natural Pharmaceuticals, Inc.

(21) Appl. No.: 11/196,950

(22) Filed: Aug. 4, 2005

Related U.S. Application Data
(63) Continuation of application No. PCT/US04/03259, filed on Feb. 3, 2004.
(60) Provisional application No. 60/444,847, filed on Feb. 4, 2003.

(51) Int. Cl. A61K 31/337; C07D 305/14

(52) U.S. Cl. 514/449; 549/510

(57) ABSTRACT

This invention relates to methods and compositions for preparing compounds using a benzoylating agent essentially free of ring chlorination. In one alternative embodiment, the present invention relates to methods and compositions for preparing taxanes essentially free of ring chlorinated impurities. In another alternative embodiment, the present invention comprises methods of converting taxane amine with a benzoylating agent essentially free of ring chlorination.

### CROSS REFERENCE TO RELATED APPLICATION

This is a continuation application of PCT/US2004/003259, filed Feb. 3, 2004, which is incorporated herein by reference in its entirety, and also claims priority from U.S. Ser. No. 60/444,847, filed Feb. 4, 2003 pursuant to 35 U.S.C. § 119. the contents of which are hereby incorporated by reference in its entirety.

### FIELD OF INVENTION

This invention relates to methods and compositions using a benzoylating agent essentially free of ring chlorination for the synthesis or semi-synthesis of compounds requiring one or more benzoyl functional groups. Such invention has clear value and utility in producing pharmaceutical compounds essentially free of ring chlorinated impurities. In one alternative embodiment, the present invention relates to methods and compositions for preparing paclitaxel and other taxanes, and in particular, to the use of a benzoylating agent essentially free of ring chlorination in the semi-synthesis of paclitaxel and its derivatives.

### BACKGROUND OF THE INVENTION

The use of a benzoylating agent essentially free of ring chlorination for the synthesis or semi-synthesis of compounds requiring one or more benzoyl functional groups has clear value and utility in producing such compounds and drugs essentially free of ring chlorinated impurities. Benzoylating agents, like benzoyl chloride, often contain undesirable amounts of ring chlorinated molecules. When such agents are used to make pharmaceutical compounds and drugs, undesirable amounts of ring-chlorinated derivatives are formed. These ring-chlorinated derivatives are impurities which can have undesirable and deleterious pharmacological, physiological and medicinal actions and properties. They can be difficult to remove from the synthesized pharmaceutical compounds and drugs. For example, taxanes, such as paclitaxel, and other compounds derived from biomass, have been identified as having significant anticancer properties. Paclitaxel can be prepared from a taxane using a benzoylating agent, e.g., benzoyl chloride, to acylate a side chain. However, benzoylating agents, like benzoyl chloride, often contain undesirable amounts of ring chlorinated molecules. Consequently, when such agents are used to make taxane compounds, such as paclitaxel, undesirable amounts of ring-chlorinated derivatives of taxane are formed. These taxane impurities are difficult to remove from the resulting taxane product. Compositions and mixtures of taxanes comprising ring chlorinated impurities have reduced utility as intermediate compounds or pharmacological agents. Therefore, there is a need for a method of producing a compound that is essentially free from ring chlorinated impurities. In at least one respect, there is a need for method of producing a pharmaceutical compound, such as paclitaxel and other taxanes for use in treating cancer, that is essentially free from ring chlorinated impurities.

### IV. SUMMARY OF THE INVENTION

Therefore, an object of the present invention is to provide a method of using a benzoylating agent essentially free of ring chlorination to make a compound essentially free of undesirable amounts of ring-chlorinated impurities.

Another object of the present invention is to make a pharmaceutical compound essentially free of undesirable amounts of ring-chlorinated impurities.

Another object of the present invention is to provide a method of making a taxane compound having a benzoyl group essentially free from chlorine atoms. The benzoyl group essentially free from chlorine may be attached to the side chain, or an amine group of the side chain, for example.

Another object of the present invention is to convert a taxane amine to paclitaxel comprising contacting the taxane amine with a benzoylating agent essentially free from ring chlorination, thus forming paclitaxel that is essentially free of ring-chlorinated derivatives thereof.

### DESCRIPTION OF THE DRAWINGS

FIG. 1 shows an example of a method for preparing paclitaxel essentially free of ring chlorination.

FIG. 2 shows an example of a method for preparing paclitaxel essentially free of ring chlorination.

FIG. 3 shows an example of the structure of paclitaxel and its ring-chlorinated derivatives.

FIG. 4 shows an example of the preparation of benzoyl chloride having small amounts of ring-chlorination.

FIG. 5 shows an example of the preparation of benzoyl chloride essentially free of ring-chlorination.

FIG. 6 shows an electrospray mass spectrum (ES-MS), for the range m/z 610 to m/z 920, of a paclitaxel compound having ring-chlorination in the benzoyl group.

FIG. 7 shows an expansion of the electrospray mass spectrum (ES-MS) shown in FIG. 6 and theoretical modeling of the spectrum.

FIG. 8 shows an example of a gas chromatogram of a commercially available benzoyl chloride containing ortho-, meta-, and para-chlorinated ring isomers.

FIG. 9 shows an example of a gas chromatogram of a benzoyl chloride essentially free of ring chlorination.

FIG. 10 shows a method of making a compound having a benzoyl group essentially free of ring chlorination.

### DETAILED DESCRIPTION OF THE INVENTION

This invention relates to methods and compositions for preparing a compound using a benzoylating agent essentially free of ring chlorination. In one alternative embodiment, the present invention is directed to a method for producing a pharmaceutical compound, such as paclitaxel comprising contacting a taxane amine with a benzoylating agent essentially free from ring chlorination. Methods and compositions for converting taxane amines to paclitaxel or other taxanes were described in Provisional Application Ser. Nos. 60/401,191 (the '191 application), filed Aug. 4, 2002; 60/370,252, (the '252 application), filed Apr. 5, 2002; and 60/370,583 (Apr. 5, 2002), filed Apr. 5, 2002. The entire disclosure of each of these applications are incorporated herein by reference in their entirety. In the present invention, the benzoylating agent may include, but is not limited to, benzoyl chloride, benzoic anhydride or benzoylating agents such as benzoic acid with one or more coupling agents. A wide variety of coupling agents may be used, including but not limited to DCC (Dicyclohexylcarbodiimide) and EDAC, and other agents known in the art.

As used herein, “taxane” or “taxane compound” means a molecule that contains a basic baccatin III structure with a (2R, 3S)—C₆H₅CH(RX)CH(OH)C)(O)— group forming an ester with the hydroxyl group located at the C-13 position of the basic baccatin III structure (where the reference numeral used to designate atom positions on a taxane molecule are in accordance with standard taxane nomenclature). The group represented by Rx can be an amino group, a salt of an amino group (e.g., an ammonium salt), an amino group which is protected with an amino protecting group (e.g., an amide), or a substituent which may be converted into an amino group. Various isomers, homologues, and analogues of the basic baccatin III structure, and to the (2R, 3S)—C₆H₅CH(RX)CH(OH)C)(O)— group are also included the definition of a taxane, For example, a 10-deacetylbaccatin III structure is contemplated within the scope of a taxane molecule. Also included within the definition of a taxane molecule are Taxol A (paclitaxel), Taxol B (cephalomannine), Taxol C, Taxol D, Taxol E, Taxol F, Taxol G, Docetaxel (TAXOTERE®), and Nonataxel. The structure of these molecules is shown in the U.S. applications cited herein, which are incorporated herein by reference in its entirety.

As used herein, the term “essentially free from ring chlorination” means that the amount of ring-chlorinated molecules in the benzoylating agent may be zero, or is less than about 700 parts per million (ppm), and is preferably less than about 500 ppm, and more preferably less than about 100 ppm, and most preferably less than about 10 ppm.

For example, in the present invention, the amount of ring-chlorinated molecules in the benzoylating agent is less than about 500, 450, 400, 350, 300, 250, 200, 150, 100, 90, 80, 70, 60, 50, 40, 30, 20 or 10 ppm. In another alternative embodiment, the amount of o-Cl, m-Cl, p-Cl derivates in the benzoylating agent is less that 100 ppm, 300 ppm and 500 ppm, respectively, preferably less than 10 ppm for each one. A taxane (e.g. paclitaxel) having a benzoyl group essentially free from ring chlorination may include any taxane (e.g., paclitaxel) produced by reacting a taxane amine with a benzoylating agent essentially free from ring chlorination, as that term is defined herein.

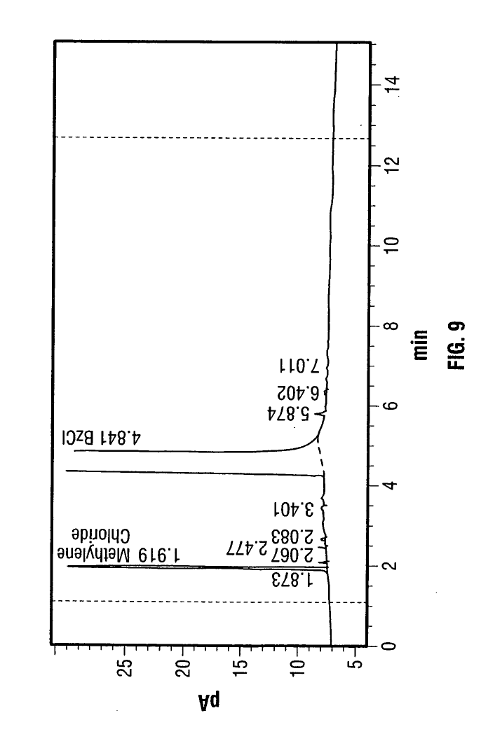

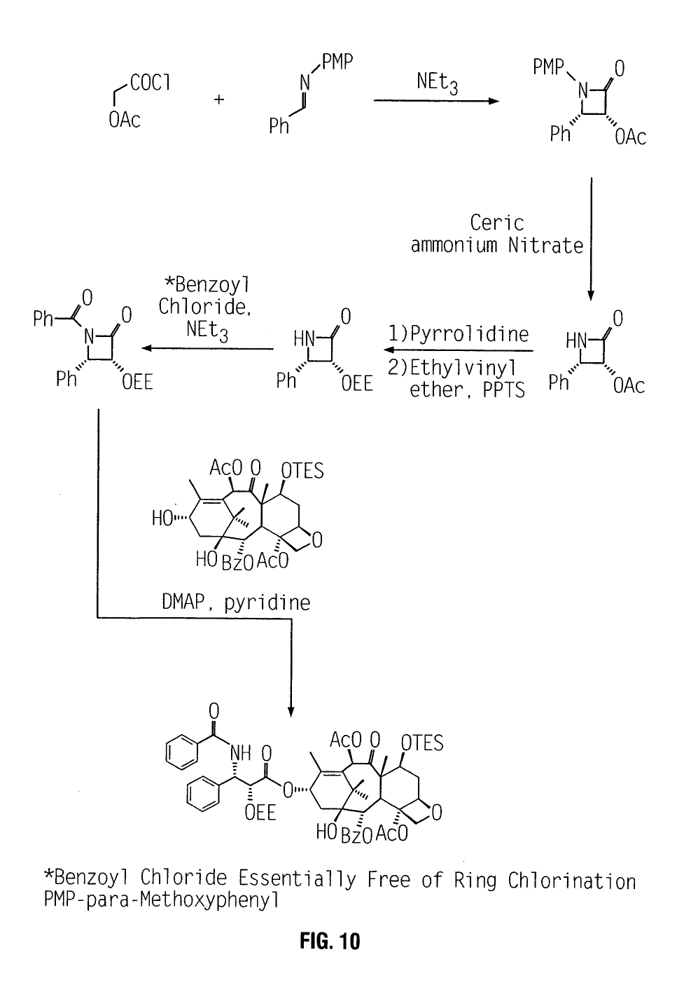

The quantity of ring-chlorinated molecules in a benzoylating agent may be measured by mass spectrometry or gas chromatography, among other methods known to those skilled in the art. A procedure for making a benzoylating agent essentially free of ring-chlorinated impurities includes, but is not limited to, treating benzoic acid with thionyl chloride, as shown in FIG. 5. This particular process is well known by those skilled in the art. Suitable benzoylating agents essentially free of ring-chlorinated impurities are commercially available from various sources, including but not limited to Soltec Ventures, Inc., of Beverly, Mass. FIG. 9 shows an example of a gas chromatogram of a benzoyl chloride essentially free of ring chlorination, which can be used in the present invention.

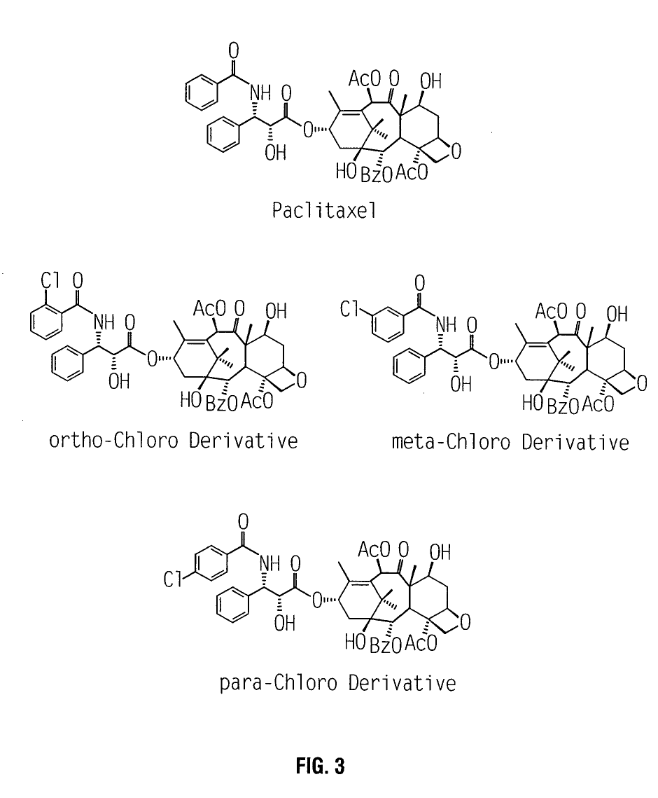

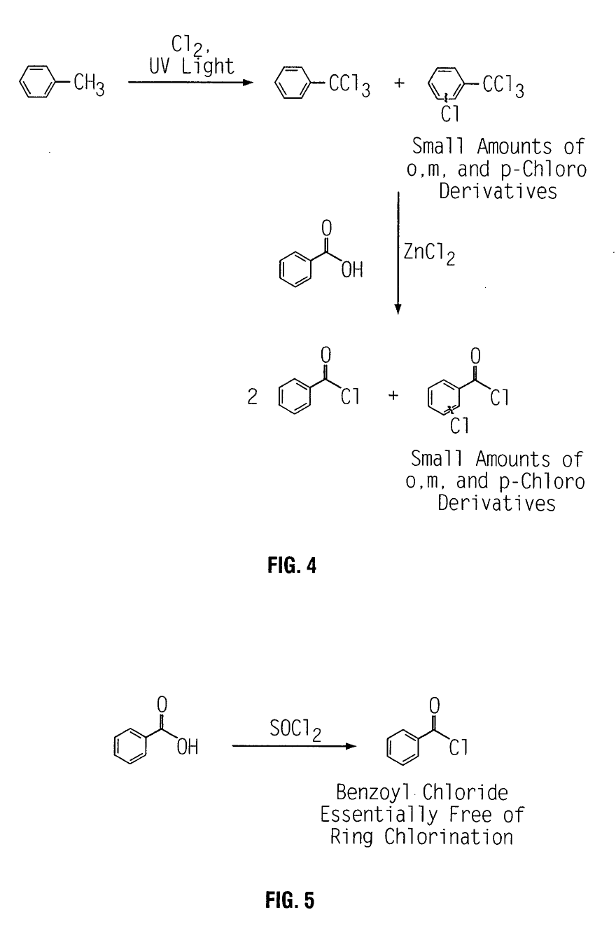

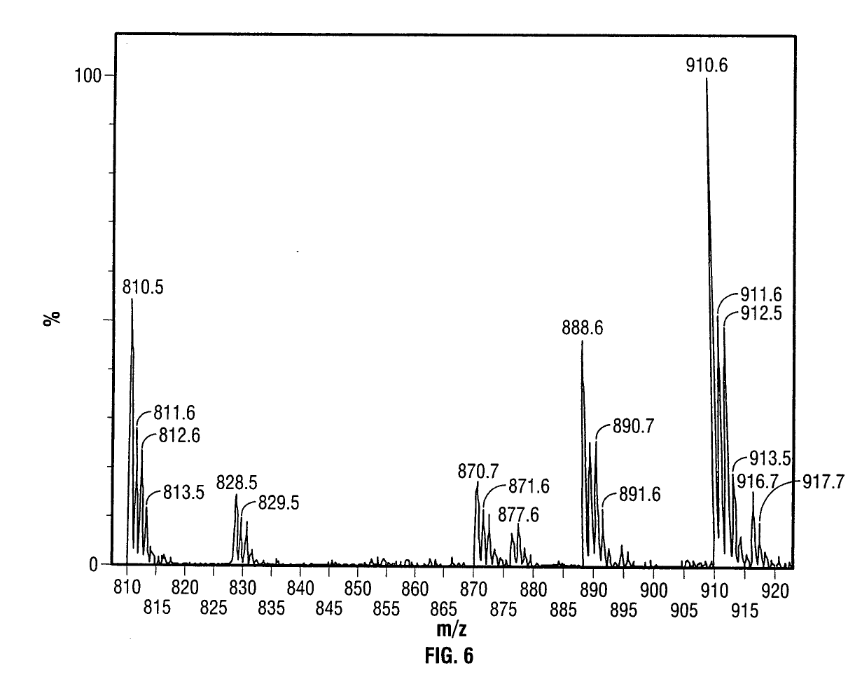

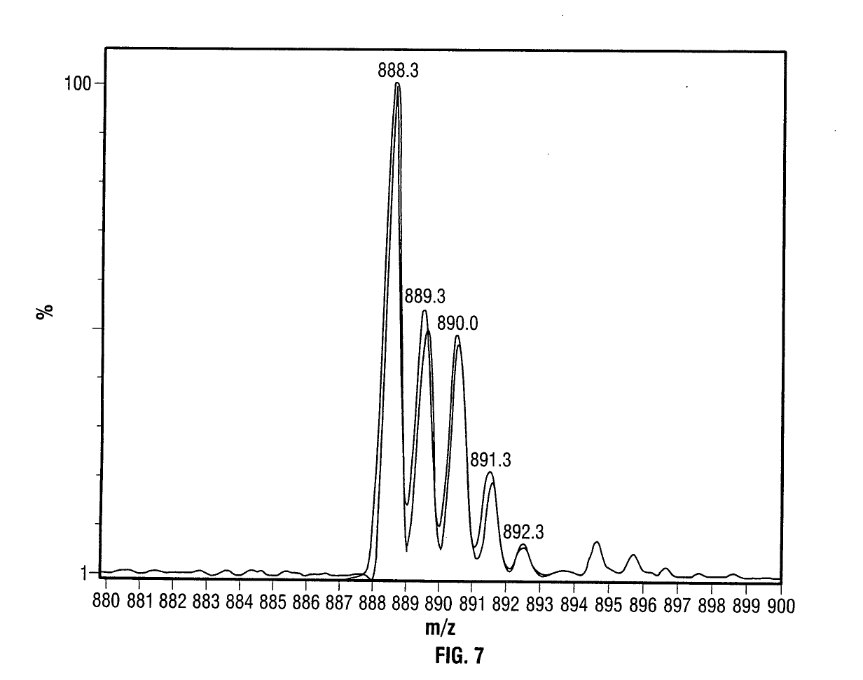

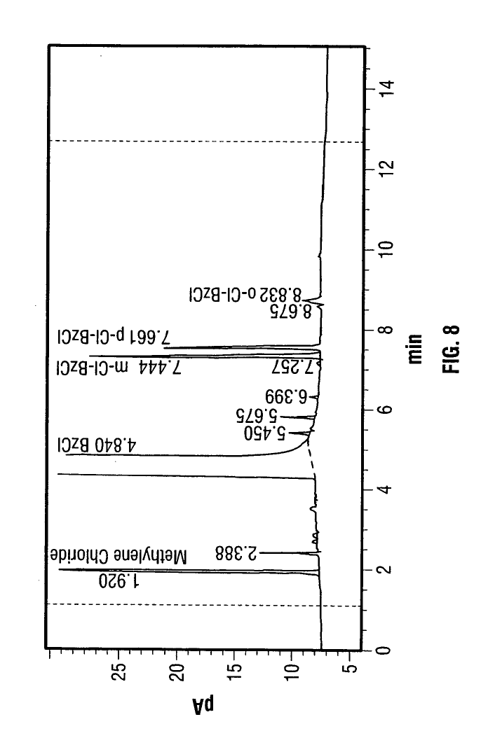

The structures of the ring-chlorinated derivatives of paclitaxel are shown in FIG. 3. Ring-chlorinated derivatives of paclitaxel may be found in conventional preparations due to the presence of ring-chlorinated impurities in some commercially available preparations of benzoyl chloride which is used to make paclitaxel, as shown in FIG. 4. FIG. 6 shows an electrospray mass spectrum (ES-MS), for the range mlz 610 to m/z 920, of a paclitaxel compound having ring-chlorination in the benzoyl group. FIG. 7 shows an expansion of the example of an electrospray mass spectrum (ES-MS) of a paclitaxel compound as shown in FIG. 6 and theoretical modeling of the spectrum. The pattern of peaks indicates that the ring-chlorinated derivatives of paclitaxel are present. FIG. 8 shows an example of a gas chromatogram of a commercially available benzoyl chloride containing ortho-, meta-, and para-chlorinated ring isomers. The peaks at retention times of 7.444 and 7.661, among others, reveal the presence of the ring-chlorinated derivatives of benzoyl chloride.

In converting taxane compounds to other taxane compounds (e.g., paclitaxel), it has been found that using benzolylating agents essentially free of ring-chlorination prevents the formation of undesirable amounts of ring-chlorinated derivatives of paclitaxel. These taxane impurities are difficult to remove from non-chlorinated taxane compounds.

Also, the use of a benzoylating agent essentially free of any cholorination advantageously provides improved selectivity for the preparation of taxanes having a benzoyl group. Improved selectivity of the reaction of the benzoylating agent with a taxane means that yield of product may be improved. Greater selectivity of the reaction also means that a taxane of higher purity may be achieved. Also, ring-chlorinated isomers of benzoyl chloride, which are impurities of benzoyl chloride, react faster with a taxane than the non-ring-chlorinated isomers of benzoyl chloride. Thus, when benzoyl chloride is used to acylate a taxane, the product taxane is inherently impure since it accumulates ring-chlorinated isomers of the benzoyl group.

1. Preparation of a Taxane Compound Using a Benzoylating Agent Essentially Free of Ring Chlorination

In one aspect of the present invention, a benzoylating agent that is essentially free from ring chlorination may be used to acylate a side chain of a taxane to introduce a benzoyl group. In one variation, a taxane may be converted to a taxane compound (e.g., paclitaxel) essentially free from ring chlorination.

In one alternative embodiment of this invention, paclitaxel may be prepared from a taxane by acylation of a taxane amine. This process employs the benzoylating agent, benzoyl chloride, to directly acylate the taxane amine group to form a benzoyl amide group in paclitaxel.

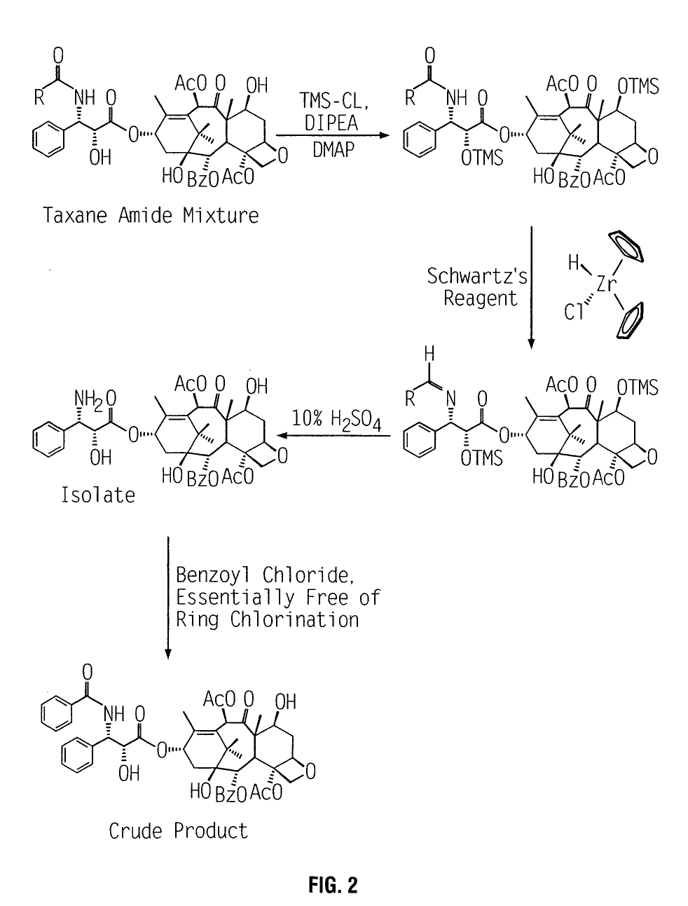

For example, a benzoyl chloride that is essentially free from ring chlorination may be used in the fourth step of FIG. 2 to prepare a crude taxane compound. Here, the benzoyl chloride is essentially free of ring chlorination, thereby producing a taxane compound that is essentially free of ring chlorination.

In one embodiment, the taxane amine may be converted into paclitaxel or other taxanes. For example, the taxane amine may be acylated to produce paclitaxel by contacting it with a benzoylating agent, preferably benzoyl chloride essentially free of ring chlorination, to produce paclitaxel or other acylated taxanes. Examples of benzoylating agents include benzoyl chloride, benzoic anhydride, or benzoic acid with one of various coupling agents such as DCC (dicarboimides), EDAC, and others known in the art.

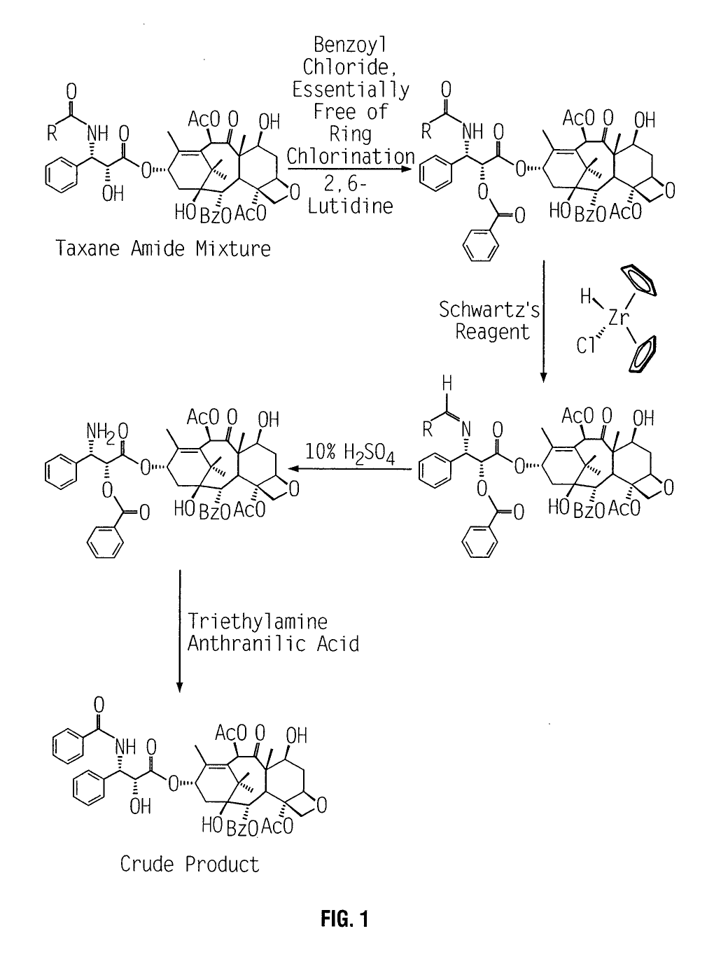

The resulting paclitaxel or other taxane compounds essentially free from ring chlorination as prepared pursuant to the reactions shown in FIGS. 1 and 2, may be further purified using various methods known in the art.

The present invention is also directed to a method of making a compound having a benzoyl group essentially free of ring chlorination. These compounds may be useful in making pharmaceutically effective compounds essentially free of ring chlorination. In one embodiment, the benzoyl group essentially free from chlorine may be attached to a compound, such as a nitrogen containing compound (e.g., an amine). This benzoyl-containing compound, or any compound obtained from it, may subsequently be attached to other compounds, e.g., a compound having a baccatin III structure, through other process steps.

In one embodiment, the present invention is directed to producing an amide or imide compound comprising contacting a compound requiring one or more benzoyl functional groups with a benzoylating agent essentially free from ring chlorination. In one embodiment, the amide compound may be a benzarnide compound.

Paclitaxel and other taxane derivatives can be semi-synthesized by synthesizing the N-benzoyl phenylisoserine side chain or a precursor thereof and attaching the compound to a taxane core such as baccatin III or 10-deacetyl baccatin III. A non-limiting example of this type of protocol is shown in Figure XYZ. This and other examples of paclitaxel side chain chemistry can be found in Taxol, Science and Applications, edited by Matthew Suffness, 1995, published by CRC Press LLC. In one embodiment, the present invention is also directed to using benzoylating agents essentially free of ring chlorination in the synthesis of the paclitaxel side chain or any precursor or derivative thereof in which the benzoyl-containing compound is used in the synthesis or semi-synthesis of paclitaxel or taxane analogue. A non-limiting example of using a benzoylating agent essentially free of ring chlorination to synthesize such a side chain or precursor thereof is shown in FIG. 9.

### EXAMPLE 1

The following is non-limiting example of a protocol for making paclitaxel essentially free of ring chlorination as shown in FIG. 1.

A 5-L flask was thoroughly dried with a heat gun and cooled by passing nitrogen through it. A taxane mixture containing 89.5% taxane molecules (consisting of 11.6% paclitaxel (taxol A), 21.4% cephalomannine (taxol B), 43.7% taxol C, 0.2% taxol D, 3.5% taxol E, 8.7% taxol F, and 0.4% taxol G) (753.2 g, 0.88 mol) was added, followed by anhydrous THF (3.0 L). Within several minutes after stirring at room temperature, the solution was homogeneous. Next, 2,6-lutidine (565 mL, 5.5 eq.) was added at once and stirred for about 2 minutes. Benzoyl chloride (510 mL, 5.0 eq.) was added over about 10 minutes (no significant exothenn was experienced). About 1 minute after the addition of the acylation reagents was complete, the stirred solution became heterogeneous. HPLC assays were performed at various intervals and at about 6 hours, the reaction was deemed complete.

The 2,6-lutidine-HC1 was filtered off through a sintered glass funnel and washed with about 500 mL of anhydrous THF. One-half of the combined filtrate was added to about 12 L of heptane over about 15 minutes, causing precipitation. This precipitate was filtered using a Buchner funnel. The other half of the filtrate was processed likewise. The collected yellow solid was washed twice with a total of about 3.75 L of a 4:1 heptane: THF solution. The yellow solid was then added to about 3 L of CH₂C1₂ and stirred for about 10 minutes to yield a crystalline solid. Then, 6 L of heptane was added over about 10 minutes with stirring. After stirring for about 30 minutes, the crystalline solid was filtered using a Buchner funnel and washed three times with a total of about 6 L of a 3:1 heptane:CH₂C1₂ solution. After drying for about 60hours under vacuum at about 40-50° C.; the crystalline solid weighed 728 grams and contained 645.9 g 2-O′-benzoyl taxane molecules (85.4% yield), and less than 0.5% unreacted taxane molecules and 2′,7-bis-O-benzoyl taxane molecules as determined by HPLC.

One kilogram of a mixture of 2′-O benzoyl taxane molecules (including taxol A, taxol B, taxol C, taxol D, taxol E, taxol F, and taxol G) was dissolved in 3.0 L anhydrous THF. The solution was added through a 1.0-micron in-line filter to a jacketed reaction vessel under an inert environment. The solution was cooled to 0-10° C. with agitation. A previously-prepared slurry consisting of 0.94 kg of Schwartz's reagent (3.2 eq.) and 2.0 L of anhydrous THF was added to the reaction solution over about 5 minutes with agitation. This reaction was mildly exothermic and generated hydrogen (H₂) gas. The reaction was agitated at 0-10° C. until the reaction end-point was determined by the HPLC assay.

When the reaction was deemed complete, the reaction mixture was added to 3.66 L of 2M aqueous bicine solution (2.0 eq. based on Zr). The reaction vessel was rinsed with 1.7 L of THF, which also was added to the bicine solution. The bicine solution was stirred at 15-20° C. for about 30 minutes. Subsequently, agitation was stopped for about 15 minutes and the lower aqueous layer was transferred to another vessel. To the aqueous layer was added 5.0 L of ethyl acetate and the solution was agitated for about 10 minutes. Agitation was stopped, for about 15 minutes allowing the layers to separate and the lower aqueous layer was removed and discarded. To the original THF solution was added another 3.66 L of 2M aqueous bicine solution along with 1.0 L of brine solution. The total solution was agitated at 15-20° C. for about 30 minutes. Agitation was stopped and the lower aqueous layer was transferred to the vessel containing the ethyl acetate. This solution was then agitated for about 15 minutes and the lower aqueous layer was discarded. The ethyl acetate was then transferred to the THF solution.

With stirring at 20-25° C., 0.4 L of a 10% aqueous H₂SO₄ solution was added to the combined organic layers and the batch stirred for about 60 minutes. Then 1.0 L of a 2M Na₂CO₃ solution was added with stirring for about 15 minutes. Agitation was stopped for about 15minutes and the lower aqueous layer was disposed of.

To the organic layer was added 0.25 kg of solid anthranilic acid and 250 mL triethylamine. The batch was agitated until the reaction end-point was determined by the HPLC assay. When the reaction was deemed complete, a total of 3.65 L of IM aqueous Na₂CO₃ solution was added with stirring for about 45 minutes at 20-25° C. Agitation was stopped for 15 minutes and the lower aqueous layer was discarded. Subsequently; 2.0 L of a saturated brine solution was added to the batch and stirred for about 15 minutes at 20-25° C. Stirring was stopped and the lower aqueous layer was discarded. To the batch was added 0.67 kg of magnesium sulfate with agitation for about 30 minutes. The 30 MgSO₄ was filtered off and the solution was added to a total of 80.0 L heptane with stirring over about 30 minutes, which resulted in precipitation. The off-white solid precipitate was filtered through a plate filter under vacuum and the solids were washed with 10.0 L of heptane. The solids were dried under vacuum at 40-50° C. for about 48 hours. The resulting mass contained about 65-75% paclitaxel which was essentially free of ring chlorination.

### EXAMPLE 2

The following is a non-limiting example of a protocol for making paclitaxel essentially free of ring chlorination as shown in FIG. 2.

(A) Small Scale

To a solution of taxane amides, 5.0 g, in 150 mL dry THF (Tetrahydrofuran) under nitrogen, 36 mg DMAP (Dimethylaminopyridine) is added, followed by 3.1 mL N-ethyl diisopropyl amine and slow addition of TMS (Trimethylsilyl) chloride, 2.1 mL, over about an hour. The reaction is stirred for another hour at room temperature. The reaction solution is then cooled to 0° C. and filtered. The salt is washed with 5.0 mL dry THF.

The filtered solution is then slowly added to a slurry of 4.69 g Bis (cyclopentadienyl) zirconium chloride hydride (Schwartz's reagent) in 100 mL dry THF (Tetrahydrofuran) under nitrogen. There is hydrogen evolution at this point, thus the reaction vessel should be large enough to accommodate some foaming. The reaction is stirred at 5° C. for 2:50 hours The reaction was 90% complete at this point.

The reaction is quenched and the Zr salts removed by adding 26.5 mL of 1M bicine solution and stirred for 37 minutes, allowing the temperature to warm to room temperature. The layers are separated and the organic layer is washed again with a second 26.5 L of 1M bicine solution as before. The layers are separated and the combined aqueous layers are washed with 5.0 mL ethyl acetate for 15 minutes. After separating the layers, the two organic layers are combined.

To the organic layers is added 3.5 mL 10% sulfuric acid solution and the solution is stirred for 45 minutes. Then 130 mL of MTBE (methyltertbutylether) is added slowly resulting in the precipitation of the primary amine taxane salt. This salt is filtered and washed with 2×12.5 mL 4:1 MTBE:THF.

The washed amine salt is then dissolved in 20 mL THF and 0.81 mL of benzoyl chloride essentially free of ring chlorination (less than about 10 ppm) is added followed by 5.0 mL saturated NaCl solution and 25 mL of phosphate buffer. The buffer is prepared by dissolving 4.72 g of potassium phosphate monobasic (KH₂PO₄) and 5.76 g of potassium phosphate dibasic (K₂HPO₄) in 300 mL of water. The reaction is stirred at room temperature for 15 minutes for reaction completion and 0.34 mL 28-30% NH₄OH solution is added and stirred for 25 minutes to neutralize excess benzoyl chloride. The aqueous layer is removed and another 0.34 mL NH₄OH is added along with 100 mL of saturated NaCl solution. After stirring for 25 minutes the aqueous layer is removed. The organic layer is then washed with 100 mL saturated NaCl solution and the aqueous layer is removed.

The organic layer is then dried with 2.5 g MgSO₄ for 10 minutes and the solution is filtered. The dried organic solution is then added slowly with stirring to 146 mL of heptane resulting in precipitation of the crude paclitaxel. The solid is then washed with 2×125 mL 4:1 heptane: THF (Tetrahydrofuran) and dried under vacuum at 45° C. for 68 hours. The yield is 4.15 g of 84.9% pure paclitaxel which is essentially free of ring chlorination. The reaction yield is 76.8%.

(B) Large Scale

To a solution of taxane amides, 500 g, in 1.5 L dry THF (Tetrahydrofuran) under nitrogen, 3.6 g DMAP (Dimethylaminopyridine) is added, followed by 307 mL N-ethyl diisopropyl amine and slow addition of TMS (Trimethylsilyl) chloride, 209 mL, over about an hour. The reaction is stirred for another hour at room temperature. The reaction solution is then cooled to 0° C. and filtered. The salt is washed with 500 mL dry THF.

The filtered solution is then slowly added to a slurry of 469 g (1.97 mol) Bis(cyclopentadienyl)zirconium chloride hydride (Schwartz's reagent) in 1 L dry THF under nitrogen. There is hydrogen evolution at this point, thus the reaction vessel should be large enough to accommodate some foaming. The reaction is stirred at 0-10° C. for 2-4 hours while monitoring by HPLC or TLC until the reaction is at least 90% complete.

The reaction is quenched and the Zr salts removed by adding 2.8 L of 1M bicine solution and stirring for 30-45 minutes, allowing the temperature to warm to room temperature. The layers are separated and the organic layer is washed again with a second 2.8 L of 1M bicine solution as before. The layers are separated and the combined aqueous layers are washed with 500 mL ethyl acetate for 15-20 minutes. After separating the layers, the two organic layers are combined.

To the organic layers is added 350 mL 10% sulfuric acid solution and the solution is stirred for 30-60 minutes. Then 10 L of MTBE is added slowly resulting in the precipitation of the primary amine taxane salt. This salt is filtered and washed with 2×1.25 L 4:1 MTBE (Methyltertbutylether): THF.

The washed amine salt is then dissolved in 2 L THF and 81 mL of benzoyl chloride essentially free of ring chlorination (less than about 10 ppm) is added followed by 500 mL saturated NaCl solution and 2.5 L of phosphate buffer. The buffer is prepared by dissolving 472 g of potassium phosphate monobasic (KH₂PO₄) and 576 g of potassium phosphate dibasic (K₂HPO₄) in 3 L of water. The reaction is stirred at room temperature for 15 minutes for reaction completion and 34 mL 28-30% NH₄OH solution is added and stirred for 20-30 minutes to neutralize excess benzoyl chloride. The aqueous layer is removed and another 34 mL NH₄OH is added along with 1 L of saturated NaCl solution. After stirring for 20-30 minutes the aqueous layer is removed. The organic layer is then washed with 1 L saturated NaCl solution and the aqueous layer is removed. The organic layer is then dried with 250 g MgSO₄ for 10 minutes and the solution is filtered. The dried organic solution is then added slowly with stirring to 14.6 L of heptane resulting in precipitation of the crude paclitaxel. The solid is then washed with 2×1.2 L 4:1 heptane: THF and dried under vacuum at 40-50° C. for 24 hours. The yield is approximately 70-85% of paclitaxel essentially free of ring chlorination.

The description and examples set forth herein are intended to illustrate representative embodiments of the invention. The claims which follow are not intended to be limited to the specific disclosed embodiments or examples. One of skill in the art will recognize that the embodiments and examples may be modified, varied or changed without departing from the scope or spirit of the invention as set forth in the following claims.

All documents referenced herein, including treatises, publications, patents and patent applications, are specifically incorporated by reference in their entirety.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously. Moreover, one or more steps or elements may be omitted from the claimed invention, or the invention described herein may be practiced in the absence of any step or element which is or is not specifically disclosed herein, so long as the invention remains operable.

We claim:

1. A method for producing a taxane comprising contacting a taxane amine with a benzoylating agent essentially free from ring chlorination to form a taxane compound.

2. The method of claim 1, wherein the benzoylating agent is benzoyl chloride.

3. The method of claim 1, wherein the benzoylating agent is benzoic anhydride.

4. The method of claim 1, wherein the benzoylating agent is benzoic acid with one or more coupling agents.

5. The method of claim 4, wherein the coupling agent is DCC or EDAC.

6. The method of claim 1, wherein the taxane is paclitaxel, its precursors or analogs thereof.

7. A compound comprising a taxane having a benzoyl group essentially free from ring chlorination made by the process of contacting a taxane amine with a benzoylating agent essentially free from ring chlorination.

8. The compound of claim 7, wherein the benzoylating agent is benzoyl chloride.

9. The compound of claim 7, wherein the benzoylating agent is benzoic anhydride.

10. The compound of claim 7, wherein the benzoylating agent is benzoic acid with one or more coupling agents.

11. The compound of claim 7, wherein the coupling agents are selected from the group consisting of DCC and EDAC.

12. The compound of claim 7, wherein the taxane is paclitaxel, its precursors or analogs thereof.

13. A method for producing a pharmaceutical compound comprising contacting a compound requiring one or more benzoyl functional groups with a benzoylating agent essentially free from ring chlorination.

14. The method of claim 13, wherein the benzoylating agent is benzoyl chloride.

15. The method of claim 13, wherein the benzoylating agent is benzoic anhydride.

16. The method of claim 13, wherein the benzoylating agent is benzoic acid with one or more coupling agents.

17. The method of claim 16, wherein the coupling agent is DCC or EDAC.

18. A reaction product comprising a pharmaceutical compound having a benzoyl group essentially free from ring chlorination made by the process of contacting a compound requiring one or more benzoyl functional groups with a benzoylating agent essentially free from ring chlorination.

19. The method of claim 18, wherein the benzoylating agent is benzoyl chloride.

20. The method of claim 18, wherein the benzoylating agent is benzoic anhydride.

21. The method of claim 18, wherein the benzoylating agent is benzoic acid with one or more coupling agents.

22. The method of claim 21, wherein the coupling agent is DCC or EDAC.

23. The benzoylating agent of claim 1, wherein the amount of ring-chlorinated molecules in the benzoylating agent is less than about 10 ppm.

24. The benzoylating agent of claim 7, wherein the amount of ring-chlorinated molecules in the benzoylating agent is less than about 10 ppm.

25. The benzoylating agent of claim 1, herein the amount of ring-chlorinated molecules in the benzoylating agent is less than 50 ppm.

26. The benzoylating agent of claim 1, wherein the amount of ring-chlorinated molecules in the benzoylating agent is less than 100 ppm.

27. The benzoylating agent of claim 1, wherein the amount of ring-chlorinated molecules in the benzoylating agent is less than 200 ppm.

28. The benzoylating agent of claim 1, wherein the amount of ring-chlorinated molecules in the benzoylating agent is less than 500 ppm.

29. The benzoylating agent of claim 7, wherein the amount of ring-chlorinated molecules in the benzoylating agent is less than 500 ppm.

30. A method of producing an amide compound comprising contacting an amine compound requiring one or more benzoyl functional groups with a benzoylating agent essentially free from ring chlorination.

31. The method of claim 29, wherein the amide compound is a benzomide compound.

32. An amide compound comprising contacting an amine compound requiring one or more benzoyl functional groups with a benzoylating agent essentially free from ring chlorination.

33. The amide compound of claim 31, wherein the amide compound is a benzomide compound.
